(19) United States
(12) Patent Application Publication
(10) Pub. No.: US 2023/0029306 A1
(43) Pub. Date: Jan. 26, 2023

(54) Method and Device for Determining the Number of Copies of a DNA Sequence That is Present in a Fluid

(71) Applicant: Robert Bosch GmbH, Stuttgart (DE)

(72) Inventor: Daniel Sebastian Podbiel, Rutesheim (DE)

(21) Appl. No.: 17/757,466

(22) PCT Filed: Dec. 17, 2020
(86) PCT No.: PCT/EP2020/086752

§ 371 Date: Jun. 16, 2022

(30) Foreign Application Priority Data
Dec. 18, 2019 (DE) 10 2019 220 020.6

(51) Int. Cl. C12Q 1/6851

(52) U.S. Cl. CPC C12Q 1/6851; C12Q 2600/16; C12Q 1/6848

(57) ABSTRACT

A method determines a number of copies of a DNA sequence that is present in a fluid. The method includes a division step, a setting up step, an identification step, and an evaluation step. In the division step, at least some of the fluid is divided into at least two compartments. In the setting up step, a reaction condition is set up for the fluid divided into the at least two compartments in order to allow a reaction in each of the at least two compartments and to obtain a reaction result in each case. In the identification step, a signal, for example an optical signal, is identified that represents the reaction results of the reactions that may have taken place in the compartments. In the evaluation step, the optical signal is evaluated in order to determine the number of copies.

### PRIOR ART

The invention is based on a method and a device for determining a number of copies of a DNA sequence contained in a fluid of the generic type of the independent claims. A computer program is also the subject of the present invention.

The amplification of target-specific DNA base sequences plays an important role in particular in the molecular-diagnostic analysis of patient samples. Since the development of the so-called polymerase chain reaction (PCR), a large number of different detection variants and amplification reactions for nucleic acids have become established.

### DISCLOSURE OF THE INVENTION

Against this background, the approach presented here provides an improved method, as well as an improved controller that uses this method and finally a corresponding computer program according to the main claims. Advantageous developments and improvements of the device specified in the independent claim are possible by the measures set out in the dependent claims.

The approach presented here allows for example an absolute quantification of a number of copies of a DNA sequence contained in a sample even when using a detection reaction with a low sensitivity. Furthermore, the approach presented here provides a possibility for advantageously using detection reactions that have a low specificity and/or a known false-positive rate in order to determine a valid test result.

The approach presented here provides a method for determining a number of copies of a DNA sequence contained in a fluid, wherein the method comprises a step of dividing at least part of the fluid into at least two partitions, which may also be referred to as compartments, reaction compartments or aliquots. The method also comprises a step of setting a reaction condition for the fluid divided into the at least two partitions/compartments, in order to allow a reaction in the at least two partitions/compartments and to obtain a reaction result for each. The method also comprises a step of detecting a strength of a signal, for example an optical signal, which represents reaction results of the reactions that have possibly taken place in the partitions/compartments. Finally, the method also comprises a step of evaluating the signal, for example the optical signal, in order to determine the number of copies while taking into account a reaction-specific detection probability function, which indicates the probability of an amplification reaction occurring in a partition/compartment in dependence on the number of copies initially present in this partition/compartment.

The detecting step may for example involve detecting an optical signal with a spatial resolution, so that the optical signal comprises or replicates information from a number of partitions/compartments.

Consequently, for example, a method for determining a number of copies of a DNA sequence contained in a fluid, which is also referred to hereinafter as the DNA target or as the gene target, comprising a dividing step, a setting step, a detecting step and an evaluating step is provided.

In the dividing step, the sample fluid, also referred to as the fluid, is divided among at least two partitions/compartments, for example by using at least one receiving unit. In the setting step, a reaction condition for the fluid divided into at least two partitions/compartments is set in order to allow and possibly cause a reaction in the at least two partitions/compartments of the fluid and thus obtain a (for example positive or negative) reaction result. In the detecting step, a signal, in particular an optical signal, which represents the reaction results of the reactions that have possibly taken place in the compartments is detected. In the evaluating step, the signal, in particular the optical signal, that is to say the reaction results of at least two compartments, is/are evaluated in order to determine the number of copies in the fluid (within the limits of statistical uncertainty).

In the setting step, a differentiation can also be made between a necessary condition, such as for example a physical condition that can be externally set for the basic occurrence of the detection reaction, and a sufficient reaction condition, such as for example DNA target molecules are present in a sufficient number of copies and are detected, wherein the setting step involves in particular creating physical ambient conditions that can be externally set and are required for the possible occurrence of a detection reaction and wherein the distribution of the DNA target molecules among the compartments takes place in particular in the dividing step.

The method may be used for example in the medical sector, for example for investigations of patient samples. The sample fluid investigated by means of the method is for example an aqueous solution, for example obtained from a biological substance, for example of human origin, such as a body fluid, a smear, a secretion, sputum, a tissue sample or a device with attached sample material. In the sample fluid there are for example species of medical, clinical, diagnostic or therapeutic relevance, such as for example bacteria, viruses, cells, circulating tumor cells, cell-free DNA or other biomarkers and/or in particular constituents from the objects mentioned. In particular, the sample fluid contains DNA molecules that have been extracted or obtained from at least one of the aforementioned species. In particular, the sample fluid is a master mix or constituents thereof, for example for carrying out at least two (mutually independent) amplification reactions in the at least two compartments, for example of at least one receiving unit, in particular for a DNA detection at molecular level by for example an isothermal amplification reaction or a polymerase chain reaction. Such a sample fluid is referred to here for example as the fluid. The necessary reaction condition represents for example an external influence that is necessary for the occurrence of a specific reaction in the fluid. The compartments may for example be provided within cavities, micro-cavities or as droplets in an immiscible second phase. Advantageously, a multiplicity of compartments make it possible for there to be more than one reaction at the same time. The signal, in particular an optical signal, for example a fluorescence signal, which emanates from the compartments and which indicates in particular the occurrence of at least one specific reaction possibly occurring in the compartments, may for example be recorded by a detection device, such as for example a sensor with spatial resolution and a light source for the optical excitation of the fluorescent probes. Advantageously, the method allows a quantification to be carried out within an extensive measuring range and/or a quantification to take place by using detection reactions with a reduced sensitivity, in particular with a detection limit really greater than one copy per compartment, which would not allow a quantitative sample analysis in a digital PCR carried out according to the prior art (a detection limit in the range of one copy per reaction compartment is required for this).

According to one embodiment, the dividing step may involve distributing at least part of the sample fluid/the fluid among at least two reaction compartments, so that partitions/aliquots of the fluid are present as reaction compartments in which mutually independent detection reactions can take place. Advantageously, this can be made possible by an automated process. For example, the partitions of the fluid may be present in cavities or micro-cavities or be realized as droplets in a second phase, such as for example an oil, and by using surfactants, which stabilize the boundary surfaces of the droplets and counteract undesired coalescing of the droplets/reaction compartments.

According to one embodiment, the distributing step may involve distributing at least part of the sample fluid/the fluid among micro-cavities, which serve for producing the reaction compartments, wherein in the micro-cavities there may be stored for example (inter alia) target-specific primers and/or probes, which can be used for detecting at least one specific DNA target. Defined storage of various target-specific primers and/or probes in predetermined micro-cavities of the device thus allows for example the sample to be investigated for different DNA targets by using a compact receiving unit. In particular, detection reactions with a restricted multiplex performance can also be used for this.

According to one embodiment, the distributing step may involve distributing at least part of the sample fluid/the fluid among micro-cavities, which serve for producing the reaction compartments, wherein the micro-cavities, and in particular the reaction compartments present in the micro-cavities, have at least two different volumes. In this way, for example, the quantification range can be further increased, since with a given concentration of a DNA target in the sample fluid the absolute number of the number of copies present in a reaction compartment is scaled with the volume of the reaction compartment. Consequently, for example—with a specific detection limit of a reaction in a compartment of for example x copies per compartment—greater DNA target concentrations in the sample fluid can also be quantitatively determined by additional use of smaller reaction compartments.

According to one embodiment, in the setting step the necessary reaction condition may represent a physical condition for the possible causing of a detection reaction. The physical condition may be for example a temperature, a temperature profile or the adding of a further fluid or substance by which advantageously a reaction can be made possible, and possibly triggered, in particular in the partitions of the fluid present in the compartments.

According to one embodiment, in the detecting step a signal, in particular an optical signal, which emanates for example from at least two reaction compartments, may be generated by means of at least one type of fluorescent probe and be detected by a detection unit. The at least one type of fluorescent probe may for example take the form of a substance which is added to the fluid and binds for example to constituents that are contained in the fluid. The binding has the effect for example of making the optical signal detectable. For example, for this purpose the fluorescent probe may initially be made up of a fluorophore and a quencher, with no detectable optical fluorescence signal being generated at first by the fluorescent probe by a Förster resonance energy transfer. Binding of the fluorescent probe to a DNA molecule can have the effect that the fluorescent probe is for example cleaved by exonuclease activity of a polymerase enzyme, so that the fluorophore and the quencher are (spatially) separate from one another and a detectable fluorescence signal is generated by the fluorophore. Advantageously, as a result the presence of a specific DNA sequence can be optically detected for example in combination with the occurrence of an amplification reaction.

According to one embodiment, the detecting step may be performed again at least one further time, in order to be able to detect a further signal, in particular a further optical signal, which represents reaction results of the reactions that have possibly taken place in at least two compartments. Advantageously, the detecting step can be performed multiple times, so that for example a plurality of measured values can be evaluated, in particular in order to be able to trace the variation over time of a (positive or negative) detection reaction on the basis of an optical signal.

According to one embodiment, the detecting step is performed multiple times, in order to detect different signals, in particular different optical signals, in particular optical signals of different wavelengths. For example, in this way various fluorescent probes can be used. In particular, at least two different fluorescent probes with different absorption and emission spectra, which indicate in particular the presence of different DNA targets in the compartment, can also be used in one reaction compartment. In this way, for example, spectral multiplexing is made possible, so that in a reaction compartment the sample fluid can be investigated for the presence of at least two different DNA targets.

According to one embodiment, between the detecting steps a time interval may be varied or be variable, in particular wherein the evaluating step may involve determining a cycle and additionally or alternatively a time interval at which a value of the optical signal, an increase in the value of the optical signal and additionally or alternatively a rate of change in the value of the increase in the optical signal can become a maximum. In this case, for example, the time interval, a temperature or the cycle may be varied in such a way that a maximum value, for example a luminosity, intensity or the like, is obtained for the optical signal. Advantageously, as a result, when using a cyclical detection reaction, for example a polymerase chain reaction, it is possible to determine a cₜ value, which correlates with the number of copies initially contained in the sample and possibly can be advantageously used for validation of the reaction result.

Furthermore, when the steps of the method are performed repeatedly, a detecting step and an evaluating step may be at least partially performed at the same time as one another. Advantageously, as a result, the progression of the reaction can be determined and/or a required period of time for determining the reaction results in the compartments, and from them the number of copies, can be reduced.

According to one embodiment, the evaluating step may involve calculating the absolute number of copies initially contained in the fluid by using the reaction results of the at least two partitions/compartments on the basis of a binomial distribution and/or by including the quantitative detection characteristic of a reaction, for example in the form of a reaction-specific detection probability function. The binomial distribution in this case includes as a general distribution function Poisson distribution and Gaussian distribution as limiting cases. The quantitative detection characteristic of a reaction in this case describes in particular the probability of the commencement of the reaction in dependence on the number of copies initially provided in the reaction compartment (and under defined boundary conditions, which are established in particular in the distributing step and/or in the setting step). Advantageously, the number of copies of at least one gene target that is initially provided in the sample fluid can thus be determined by using a known reaction-specific detection probability function with statistical significance.

This method may for example be implemented in software or hardware or in a mixed form of software and hardware, for example in a controller.

The approach presented here also provides a controller which is designed to carry out, activate or implement the steps of a variant of a method presented here in corresponding devices. This variant of an embodiment of the invention in the form of a controller also allows the object on which the invention is based to be achieved quickly and efficiently.

For this purpose, the controller may have at least one computing unit for processing signals or data, at least one memory unit for storing signals or data, at least one interface with respect to a sensor or an actuator for reading in sensor signals from the sensor or for outputting control signals to the actuator and/or at least one communication interface for reading in or outputting data, which are embedded in a communication protocol. The computing unit may be for example a signal processor, a microcontroller or the like, wherein the memory unit may be a flash memory, an EEPROM or a magnetic storage unit. The communication interface may be designed to read in or output data in a wireless and/or line-bound manner, wherein a communication interface which can read in or output line-bound data can read in these data for example electrically or optically from a corresponding data transmission line or output them into a corresponding data transmission line.

A controller may be understood in the present case as meaning an electrical device which processes sensor signals and, in dependence thereon, outputs control and/or data signals. The controller may have an interface, which may be formed on the basis of hardware and/or software. In the case of a hardware-based form, the interfaces may for example be part of a so-called system ASIC, which comprises a wide variety of functions of the controller. It is however also possible that the interfaces are dedicated, integrated circuits or at least partially consist of discrete components. In the case of a software-based form, the interfaces may be software modules, which are for example present on a microcontroller along with other software modules.

In an advantageous configuration, the controller is responsible for controlling a method for determining a number of copies of at least one DNA sequence contained in a fluid. For this purpose, the controller may for example access sensor signals such as a setting signal for setting a reaction condition and an optical signal, which represents the reaction results of the reactions that have possibly taken place in the compartments.

The activation takes place by way of actuators such as a setting unit, which is designed to output the setting signal, and a detection unit, which is designed to detect the optical signal.

Also of advantage is a computer program product or computer program with program code, which can be stored on a machine-readable carrier or storage medium such as a semiconductor memory, a hard-disk storage unit or an optical storage unit and is used for carrying out, implementing and/or activating the steps of the method according to one of the embodiments described above, in particular when the program product or program is run on a computer or a device.

Exemplary embodiments of the approach presented here are explained in more detail in the following description and are represented in the drawings, in which:

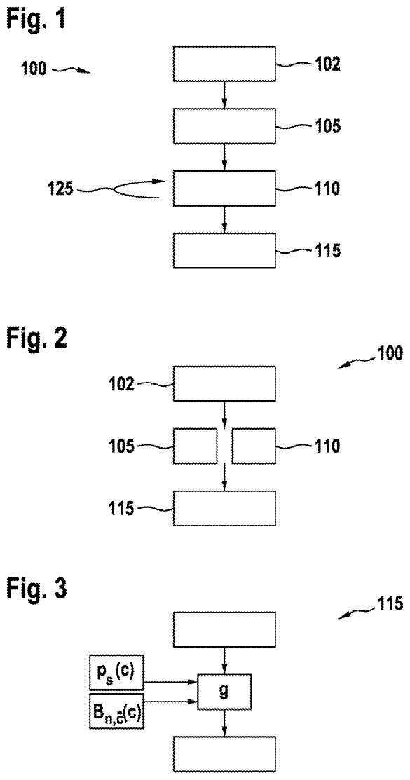

FIG. 1 shows a flow diagram of an exemplary embodiment of a method for determining a number of copies of a DNA sequence contained in a fluid;

FIG. 2 shows a flow diagram of an exemplary embodiment of a method for determining a number of copies of a DNA sequence contained in a fluid;

FIG. 3 shows a flow diagram of a step for evaluating a method for determining a number of copies of a DNA sequence contained in a fluid according to an exemplary embodiment;

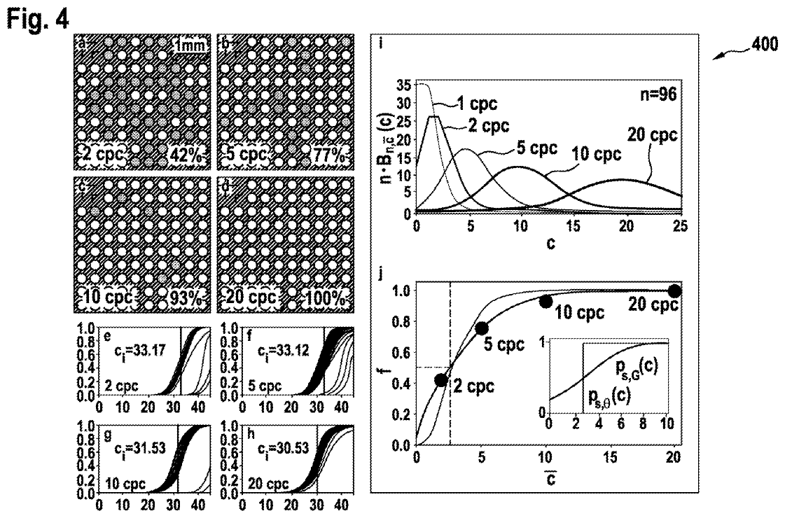

FIG. 4 shows a schematic representation of a series of measurements according to an exemplary embodiment that is carried out by means of a method for determining a number of copies of a DNA sequence contained in a fluid; and

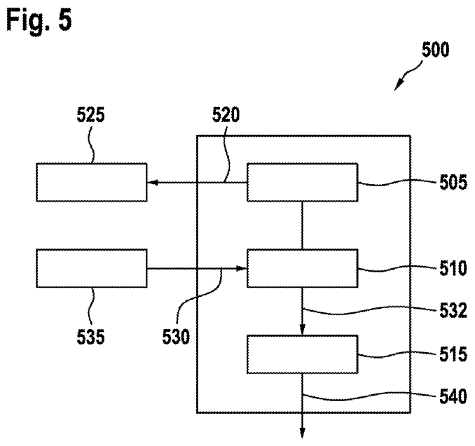

FIG. 5 shows a block diagram of an exemplary embodiment of a controller.

In the following description of favorable exemplary embodiments of the present invention, the same or similar designations are used for the elements that are presented in the various figures and act in a similar way, without the description of these elements being repeated.

FIG. 1 shows a flow diagram of a method 100 for determining a number of copies of at least one DNA sequence contained in a fluid according to an exemplary embodiment. The method 100 can be used for example in the area of molecular laboratory diagnostics. The method 100 can for example be activated by a controller, such as that described in one of the following figures.

In a step 102 of the method 100, dividing of at least part of the fluid into at least two partitions/compartments takes place. The method 100 comprises a further step 105 of setting a necessary reaction condition for the fluid divided into the at least two partitions/compartments, in order to allow a reaction in the at least two partitions/compartments and to obtain a reaction result for each. In a detecting step 110, a strength of a signal is detected, for example an optical signal, which represents reaction results of the reactions that have possibly taken place in the compartments. In a step 115 of evaluating the signal, the signal is evaluated. Evaluation takes place while taking into account the statistical distribution of the numbers of copies in the compartments and by using a reaction-specific detection probability function, which indicates the probability of the occurrence of an amplification reaction in the compartments in dependence on the number of copies initially present in the compartments. In this way, the number of copies of at least one target/DNA sequence initially provided in the fluid can be determined with statistical accuracy on the basis of the reaction results achieved in the compartments.

The determination of a quantitative reaction result therefore takes place in this case on the basis of a statistical evaluation of at least two (mutually independent) detection reactions. In order to achieve a quantification that is as accurate as possible in a great measuring range, generally a multiplicity of compartments is favorable, typically more than 10, better 50 to 1000 or even 10 000 to 100 000. The number of compartments is scaled with the quantification range; depending on how great it is intended to be, a correspondingly great number of mutually independent reaction compartments are required.

According to this exemplary embodiment, the dividing step 102 is carried out before the setting step 105. The first step of distributing/partitioning/aliquoting the fluid/the sample fluid is the basis here for the subsequent evaluation. A “compartment” or “reaction compartment” is understood in this connection as meaning a restricted/delimited volume of fluid in which a detection reaction can possibly take place. The production of compartments may for example take place within micro-cavities or else also by the generation of droplets in a second immiscible fluid. For the reaction compartments to be generated in micro-cavities, the micro-cavities may in particular first be filled with the sample fluid by way of an adjoining channel and then be sealed with a second fluid that cannot be mixed with the sample fluid, for example an oil, wherein the sample fluid is displaced (completely) from the region adjoining the micro-cavities.

The partitioning or dividing of the fluid is characteristic of the method presented here; the quantification takes place in particular by counting off the positive/negative reactions in the compartments.

According to this exemplary embodiment, the reaction condition represents for example a physical condition, such as for example a temperature or a temperature profile, whereby for example a reaction in the partition/compartment can be made possible. It should be noted here that generally the specific detection reaction particularly only takes place when there is in a compartment at least one molecule that can be detected by the reaction. Otherwise, there is a false-positive reaction result in a compartment.

The reaction result in a reaction compartment is determined for example by means of an optical signal, for example by means of a fluorescent probe. The fluorescent probe is realized for example as a substance which for example can bind itself to another substance in the fluid and as a result makes the reaction result detectable. According to this exemplary embodiment, the renewed detection is symbolized by means of an arrow 125. According to this exemplary embodiment, a time interval between the detecting steps 110 is also optionally varied or variable. Furthermore, in the evaluating step 115, a cycle and/or time interval at which a value of the optical signal, an increase in the value and/or a rate of change in the value becomes a maximum can be determined. When using a fluorescent dye with a temperature-dependent fluorescence, in this way for example a tracking of temperature cycles—for example in conjunction with the carrying out of polymerase chain reactions—can be achieved. In this way, in addition to the function of detecting a reaction in a compartment, the optical signal can also be used for checking the temperature profile in a compartment, and consequently in particular for checking the setting of a necessary reaction condition.

According to this exemplary embodiment, in the evaluating step 115, the absolute number of copies of at least one DNA sequence initially contained in the fluid (the expected value of the number of copies) is calculated by using the reaction results of the reactions possibly occurring in the individual compartments, generally on the basis of a binomial distribution. The binomial distribution in this case includes as a general distribution function Poisson distribution and Gaussian distribution as limiting cases. As a result—when using a detection reaction with reduced sensitivity, in particular with a detection limit, i.e. a limit of detection (LOD), really greater than 1—a calculation of the number of copies of at least one DNA sequence is also made possible when there are multiple copies of the DNA sequence initially present in a detection compartment.

In other words, a possibility for quantitative DNA analytics is provided on the basis of a detection-reaction-specific amplification characteristic.

Digital PCR represents a variant that has been used so far. In the case of digital PCR, a PCR master mix, which contains at least one fluorescent probe and the sample material to be analyzed, is first divided among a multiplicity of spatially separate, i.e. mutually independent, reaction compartments. After thermocycling of the reaction compartments, it is determined on the basis of the fluorescence signal in which reaction compartments an amplification has taken place. By simply counting off the positive (and negative) reactions, the amount of target-specific DNA initially present in the sample can subsequently be quantified absolutely on the basis of Poisson statistics.

The quantification based on Poisson statistics in digital PCR is in this case based on highly sensitive PCR detection reactions, which can already reliably detect the presence of an individual DNA target molecule in a reaction compartment. The sensitivity (so-called limit of detection, LOD) of a detection reaction may however be lower, and generally competes with the specificity, that is to say the accuracy with which a specific target can be reliably detected. If the specificity of the detection reaction is too low, this can lead to false-positive results. Therefore, a suitable compromise between the sensitivity and the specificity of the reaction must generally be found in the design of a detection reaction (for example primer design). The presetting of a very high specificity of a detection reaction is possibly not compatible with a very high sensitivity in the range of a single copy.

By contrast with the approach used so far, with the newly presented approach there may be multiple copies of a DNA sequence in a reaction compartment, and the number of copies of the DNA sequence initially present in the fluid can be inferred in a statistical way on the basis of a “quantitative amplification characteristic of a detection reaction”. The “quantitative amplification characteristic of a detection reaction” in this case describes the probability of the occurrence of an amplification reaction for the detection of a DNA sequence in dependence on the number of copies of the DNA sequence to be amplified by the reaction that are initially present in the reaction compartment. The statistical calculation may take place in particular by means of the binomial distribution.

According to this exemplary embodiment, presented for this purpose is the method 100, which allows an absolute quantification of a DNA sequence/target DNA in a sample, which is referred to here as the fluid or sample fluid, even in the case of a reduced sensitivity, which means in the case of a so-called limit of detection (LOD)>1 of the detection reaction. Furthermore, according to this exemplary embodiment, the method 100 takes into account a general detection characteristic of an amplification reaction with respect to sensitivity and specificity (that is to say in particular also possibly including a false-positive rate), in particular commencement behavior of the amplification reaction in order to use it to determine a valid test result.

Therefore presented is the method 100, which in the introducing step 102 makes it possible for a fluid with sample material contained therein, which is referred to here as the fluid, to be divided among a large number of reaction compartments, which may also be referred to as compartments and may for example be present in micro-cavities. According to this exemplary embodiment, the method 100 comprises the setting step 105 for establishing suitable physical conditions, such as for example the temperature or temperature profile, in the compartments, which for example allow the occurrence of amplification reactions in these. In the detecting step 110, a detection of the reaction results in the individual compartments is carried out for example by an optical signal, which is caused by a fluorescent probe. It is also noted in this respect that from each individual compartment there emanates an optical signal, which indicates the reaction result in the compartment. The “optical signal” mentioned here then comprises the plurality of optical signals that emanate from the individual compartments. In the evaluating step 115, a statistical evaluation of the reaction results in multiple compartments is carried out on the basis of the binomial distribution as a general distribution function with the limiting cases of Poisson distribution and Gaussian distribution, for example with the inclusion of a quantitative detection reaction characteristic, in particular by using a quantitative description of the commencement behavior of the detection reaction, which means in particular while taking into account the sensitivity and specificity (that is to say in particular also possibly including a false-positive rate) of the detection reaction. Furthermore, a statistically verified test result is derived, and the absolute number of copies initially provided in the fluid, for example of at least one DNA sequence, is possibly calculated with statistical significance.

Advantageously, a large number of given detection reactions can thereby be used for an absolute quantification of DNA copies of at least one gene target that are initially present in a sample fluid. In particular, a lower sensitivity of the detection reactions, that is to say a limit of detection really greater than one, is also sufficient. In particular, detection reactions which are distinguished by a higher specificity and lower sensitivity can be used for a quantification. In comparison for example with a digital PCR according to the prior art, which is limited to the range described by Poisson statistics, the method 100 described here, which is based on the more general binomial statistics, can be used to achieve quantification within a different measuring range, possibly with the use of the same aliquoting device. According to this exemplary embodiment, however, this is dependent on the sensitivity characteristic of the amplification reaction. By combining differently designed detection reactions with different sensitivity and/or specificity for a gene target, quantification can advantageously be carried out within a larger measuring range. Likewise, according to this exemplary embodiment, on the basis of the method 100 presented here, detection reactions with a low specificity and a known significant false-positive rate can also be used to determine a valid test result. By aliquoting the sample fluid among a large number of compartments and performing (almost) independent amplification reactions on the basis of an experimentally determined proportion of positive reactions, including a known reaction-specific false-positive rate, inferences can be made about the actual composition of the sample with statistical significance.

In the basic embodiment, the method 100 presented here comprises the steps 102, 105, 110, 115. In the step 102 of the method 100, the fluid with the sample material contained therein is divided among a large number of reaction compartments. In particular, the fluid contains nucleic acids. According to this exemplary embodiment, in particular the compartments all have the same volume. In the step 105 of the method 100, suitable physical conditions, such as the temperature or temperature profile, that allow amplification reactions to take place in them are established in the compartments. In particular, these are nucleic acid-based methods, such as for example the polymerase chain reaction or an isothermal amplification method. In the step 110 of the method 100, the reaction result is detected in the individual compartments, for example on the basis of an optical signal which is produced by at least one fluorescent probe. For example, a quantitative polymerase chain reaction can be used as the detection reaction by using a master mix with a target-specific fluorescent probe which indicates the presence of a specific PCR product. In this way, the reaction kinetics can be followed in real time on the basis of a fluorescence signal (an increase in it). In the step 115 of the method 100, a statistical evaluation of the reaction results takes place in multiple compartments. In particular, the evaluation takes place on the basis of the binomial distribution as a general distribution function with the limiting cases of the Poisson distribution and the Gaussian distribution and with the inclusion of the quantitative characteristic of the detection reaction. This means in particular by using the commencement behavior of the reaction with regard to sensitivity and specificity. A statistically verified positive or negative test result is derived from it; optionally, a calculation of the absolute number of copies of at least one DNA sequence/gene target initially provided in the sample fluid with statistical probability is carried out. If, for example, a quantitative polymerase chain reaction is used as the detection reaction, the amount of DNA initially present in the sample can also be inferred from an optional comparison of the reaction kinetics in the individual compartments with standard reactions (which take place with a defined initially present number of copies) and be combined with the statistically determined test result on the basis of the reaction compartments.

FIG. 2 shows a flow diagram of a method 100 for determining a number of copies of a DNA sequence contained in a fluid according to an exemplary embodiment. The method 100 shown here can correspond or be similar to the method 100 described in FIG. 1. Only the steps 105, 110 are shown differently, since according to this exemplary embodiment they can be carried out in parallel. This means that, according to this exemplary embodiment, when the steps of the method 100 are performed repeatedly, a setting step 105 and a detecting step 110 can be at least partially performed at the same time as one another. According to this exemplary embodiment, the steps 102, 115 can still be performed unchanged.

This exemplary embodiment also presents the method 100, which allows the determination of the absolute number of copies of at least one DNA sequence present in the fluid, while a detection reaction with a reduced sensitivity, that is to say a limit of detection (LOD), really greater than one can be used for this. Furthermore, it also allows a valid, possibly quantitative test result to be derived by using detection reactions with limited sensitivity and specificity which, taken by themselves, do not produce a valid test result.

In other words, according to this exemplary embodiment, step 105 and step 110 are performed in parallel, that is to say the detection of the fluorescence signal takes place at a number of times when the amplification reaction is being carried out. As a result, the progression of the reaction can additionally be determined, and this can allow even more reliable detection of positive and negative detection reactions. In particular, according to this exemplary embodiment, in a quantitative polymerase chain reaction, for example, the cycle at which the increase in the fluorescence signal or the rate of change in the increase in the fluorescence signal becomes a maximum (“cₜ value”) can also be determined. Since this value likewise correlates with the initial number of copies contained in the fluid, it can possibly also be used to validate the test result.

FIG. 3 shows a flow diagram of an evaluating step 115 of a method for determining a number of copies of a DNA sequence contained in a fluid according to an exemplary embodiment. The evaluating step 115 may correspond to the evaluating steps 115 described in one of FIG. 1 or 2.

In step 115, the absolute number of copies initially present in the fluid is calculated in particular on the basis of the reaction result from the detecting step of the method, that means for example a measured positive rate, and by using a predetermined function g, which takes into account the quantitative characteristic of the commencement behavior of the detection reaction pₛ(c) and the statistical distribution of the sample DNA among the compartments B_{n,c̅}(c).

The text which follows describes in more detail the determination of the function g, which allows the calculation of the amount of DNA of a gene target initially provided in a sample on the basis of the measured positive rate for a specific detection reaction under specific boundary conditions with statistical significance. In particular, to provide a function g, the quantitative characteristic of a detection reaction in a given microfluidic compartment is first described (approximately) by a function pₛ(c), which can also be referred to for example as a detection probability function, probability-of-detection (POD) function or as a “sensitivity characteristic of a detection reaction” (at least for a relevant measuring range), which indicates the probability that, if exactly c copies are present in a compartment, an amplification reaction will take place in this compartment. For a (simplified) approximative description, for example the Heaviside function Θ may be used here, so that

p_{s,Θ,LOD}(c)=Θ(c−c_{LOD})

where c_{LOD} indicates the limit of detection (LOD) of the detection reaction. In general, more complicated functions are also suitable for the quantitative characterization of the commencement behavior of an amplification reaction pₛ(c), such as for example polynomials which have been determined on the basis of a large number of experimental data records and thus map the assay characteristic even more precisely in the test setup used. A further (approximative) description results, for example, from the convolution of the above Heaviside function p_{s,Θ,LOD}(c) with a Gaussian function

$${G}_{w , {c}_{0}} ( c ) = \frac{1}{\sqrt{2 π {w}^{2}}} {e}^{- {( c - {c}_{0} )}^{2} / 2 {w}^{2}}$$

of the width w, so that, depending on the number of copies c initially present in a compartment, a continuous commencement of the amplification reaction can be mapped by the function

$${p}_{s , G , LOD , w} ( c ) = {\int }_{- ∞}^{∞} {dc}^{′} {p}_{s , Θ , LOD} ( c - {c}^{′} ) {G}_{w , {c}_{0} = 0} ( {c}^{′} )$$

With a number of compartments n and an average number of initial copies per compartment c̅, the following binomial distribution B_{n,c̅}(c) is obtained, describing the proportion of compartments in which there are initially exactly c copies:

$${B}_{n , \overline{c}} ( c ) = ( \begin{matrix}n \cdot \overline{c} \\ c\end{matrix} ) {{n}^{- c} ( 1 - 1 / n )}^{n \cdot \overline{c} - c}$$

The function pₛ(c) introduced above, for the quantitative description of the amplification characteristic, then results in the proportion f of compartments in which a positive detection reaction i takes place

f=∫₀^{∞}dc′B_{n,c̅}(c′)·pₛ(c′)

With the approximative Heaviside description of the commencement of the amplification reaction p_{s,Θ,LOD}(c) there follows the formula

f=∫_{c}_{_{LOD}}^{∞}dc′B_{n,c̅}(c′)

so that f=f(n,c̅,c_{LOD}) For the Gaussian description it correspondingly follows that f=f(n,c̅,c_{LOD},w) According to these (approximate, empirical) descriptions of the reaction characteristic, the proportion of compartments f in which an amplification reaction takes place depends directly on the average number c̅ of initial copies per compartment and the, for example empirically known, limit of detection c_{LOD} of the detection reaction and also possibly the width of the commencement w. Accordingly, in the case of an unknown sample on the basis of the measured positive rate f, and in the case of a known c_{LOD} (and possibly a known w), inferences can be drawn about the initial average number of copies per compartment c̅, and consequently the absolute number of copies in the sample can be determined, as long as there is at least in a partial area/interval a monotony of the function g(f,n,c_{LOD},w)=c̅ with respect to a change in f.

With the continuous description by means of integral terms chosen in the previous paragraph, the binomial coefficients can be described by means of the beta function. In addition to the continuous representation, a discrete description can also be used throughout, so that

$$f = {\sum }_{c = 0}^{∞} {p}_{s} ( c ) \cdot {B}_{n , \overline{c}} ( c )$$

and the other formulas are obtained analogously.

FIG. 4 shows a schematic representation of a series of measurements 400 carried out by means of a method for determining a number of copies of a DNA sequence contained in a fluid, according to an exemplary embodiment. The reaction results of the series of measurements 400 shown here, inter alia with the aid of curve diagrams, can be generated for example by means of a method as explained in one of FIGS. 1 to 3 described above.

In other words, according to this exemplary embodiment, an exemplary experimental series of measurements 400 from schematic representations of fluorescence micrographs that were made in the detecting step is shown inter alia. This involved using a PCR detection reaction by using target-specific primers and a fluorescent probe for a diagnostically relevant gene target. In each of the batches there was a defined amount of template DNA that contains the gene target. The average numbers of copies per compartment c̅ were 2, 5, 10 and 20 copies per compartment (cpc). In the schematic representations of fluorescence micrographs in FIGS. 4(a)-(d), which were made after thermocycling, the reaction compartments in which amplification took place appear light, while the others appear dark. The associated quantitative PCR amplification curves are likewise shown schematically in FIGS. 4 (e)-(h). The positive rate of the detection reactions extends from 42% with c̅=2 initial copies per compartment (cpc), through 77% with 5 cpc and 93% with 10 cpc, to 100% with 20 cpc (see FIGS. 4 (a)-(d)). By including the average initial number of copies c̅ as well as the binomial statistics (see FIG. 4 (i) for the illustration of the distribution functions when using 96 compartments and average numbers of copies per compartment of 1, 2, 5, 10 and 20 copies per compartment, cpc), the sensitivity characteristic pₛ(c) of the detection reaction can be inferred from the experimentally determined positive rates f on the basis of the measurement series 400:

For this purpose, FIG. 4 (j) shows a plot of the experimentally determined positive rates (measuring points) and the calculated positive rates (curves) obtained from modeling the commencement behavior of the amplification reaction by means of the Heaviside description (inset, thin line) and Gaussian description (inset, thick line) by using suitable parameters that are characteristic of the amplification reaction, plotted against the average number of copies per compartment. The curves depicted in FIG. 4 (j) show the calculated positive rates obtained for the parameters c_{LOD}=2.6 (Heaviside commencement, thin line) and c_{LOD}=2.5, w=2.95 (Gaussian commencement, thick line).

Particularly when using the Gaussian description, good agreement with the experimentally determined positive rates can be achieved. Accordingly, the commencement of the amplification reaction in the range of average initial numbers of copies per compartment c̅ of between 2 and 20 can be quantitatively mapped. Conversely, by using the obtained quantitative description of the commencement behavior of the amplification reaction, the number of copies initially present in a sample fluid can be determined from an experimentally determined positive rate.

FIG. 5 shows a block diagram of a controller 500 according to an exemplary embodiment. According to this exemplary embodiment, the controller 500 has a setting unit 505, a detecting unit 510 and an evaluating unit 515. The setting unit 505 is in this case designed to provide a setting signal 520 to for example a setting device 525, which for example takes the form of a heating device and/or cooling device, in order to allow a reaction in the at least two partitions/compartments and to obtain a reaction result. The detecting unit 510 is designed to detect an optical signal 530, which represents the reaction results 532 of the reactions that have possibly taken place in the partitions/compartments. The optical signal 530 can in this case be detected for example by a sensor device 535. The evaluating unit 515 is designed to evaluate the optical signal 530 and/or the reaction results 532 and to determine the number of copies from it/them. The number of copies can be shown graphically in a diagram, for example as an evaluation result 540. For example, the number of copies determined is of medical, clinical, diagnostic or therapeutic relevance, so that, depending on the number of copies determined and possibly with the inclusion of further information, a patient can be treated.

If an exemplary embodiment comprises an “and/or” conjunction between a first feature and a second feature, this should be read as meaning that, according to one embodiment, the exemplary embodiment comprises both the first feature and the second feature and, according to a further embodiment, the exemplary embodiment comprises either only the first feature or only the second feature.

Exemplary specifications for the method according to the invention are given below:

Number of reaction compartments:

2 to 1 000 000, preferably 10 to 30 000

Volume of a reaction compartment:

5 μl to 100 μl, preferably 500 μl to 1 μl

Detection reaction:

An isothermal amplification reaction or a (quantitative) polymerase chain reaction

1. A method for determining a number of copies of a DNA sequence contained in a fluid, the method comprising:
dividing at least a predetermined part of the fluid into at least two compartments;
setting a reaction condition for the fluid divided into the at least two compartments, in order in each case to allow a reaction in the at least two compartments and to obtain a reaction result for each;
detecting a strength of a signal, which represents the reaction results of the reactions that have taken place in the at least two compartments; and
evaluating the signal, in order to determine the number of copies, based on a reaction-specific detection probability function, which indicates a probability of an amplification reaction occurring in a compartment of the at least two compartments in dependence on the number of copies initially present in the compartment of the at least two compartments.

2. The method as claimed in claim 1, wherein the evaluating the signal comprises:
using a binomial distribution function for a statistical description of a distribution of the initially present copies among the at least two compartments for the determination of the number of copies.

3. The method as claimed in claim 1, wherein the detecting the strength of the signal comprises:
detecting the strength of an optical signal.

4. The method as claimed in claim 1, wherein the evaluating the signal comprises:
investigating the fluid for multiple DNA sequences.

5. The method as claimed in claim 1, wherein the setting the reaction condition comprises:
introducing at least one additional reactant into the fluid.

6. The method as claimed in claim 1, wherein the setting the reaction condition comprises:
setting the reaction condition at least partially only after the dividing.

7. The method as claimed in claim 1, wherein the evaluating the signal comprises:
using an amplification reaction which has a detection limit which really is greater than 1 copy per reaction compartment.

8. The method as claimed in claim 1, wherein the detecting the strength of the signal comprises:
recording spectral information of an optical signal.

9. The method as claimed in claim 1, further comprising:
detecting the strength of the signal again at least one more time, in order to detect at least one further signal and to determine from the detected signals the reaction results of the reactions that have taken place in the at least two compartments using the signals.

10. The method as claimed in claim 9, further comprising:
varying, between the detecting the strength of the signals, a time interval,
wherein the evaluating the signals includes determining a cycle, a temperature, and/or a time interval at which a value of an optical signal, an increase in a value of the optical signal, and additionally or alternatively a rate of change in the value of the increase in the optical signal becomes a maximum.

11. The method as claimed in claim 1, further comprising:
performing the method repeatedly; and
at least partially performing at the same time the setting the reaction condition and the detecting the strength of the signal.

12. The method as claimed in claim 1, wherein the dividing at least the predetermined part of the fluid comprises:
using a receiving unit with cavities.

13. The method as claimed in claim 1, wherein a controller is configured to perform and/or activate the method.

14. The method as claimed in claim 1, wherein a computer program is configured to perform and/or activate the method.

15. The method as claimed in claim 14, wherein the computer program is stored on a non-transitory machine-readable storage medium.
